(19) United States
(12) Patent Application Publication
(10) Pub. No.: US 2014/0112964 A1
(43) Pub. Date: Apr. 24, 2014

(54) COMPOSITIONS COMPRISING HYDROGEL PARTICLES

(71) Applicant: Xufeng Wu, Shanghai (CN)

(72) Inventor: Xufeng Wu, Shanghai (CN)

(73) Assignee: JOHNSON & JOHNSON CONSUMER COMPANIES, INC., Skillman, NJ (US)

(21) Appl. No.: 13/818,743

(22) PCT Filed: May 3, 2012
(86) PCT No.: PCT/US12/36346

§ 371 Date: Oct. 29, 2013

(30) Foreign Application Priority Data
May 6, 2011 (CN) 201110126994.8

(51) Int. Cl. A61K 8/73; A61K 8/04

(52) U.S. Cl. CPC A61K 8/73; A61K 8/042
USPC 424/401; 514/54

(57) ABSTRACT

Provided are stable compositions comprising an aqueous carrier, hydrogel particles comprising one or more polysaccharides, and one or more surfactants. Also provided are methods of making such compositions.

### FIELD OF INVENTION

The present invention relates to aqueous compositions and methods of their preparation. In particular, the present invention relates to stable aqueous compositions having dispersed therein hydrogel particles comprising polysaccharides.

### DESCRIPTION OF RELATED ART

A variety of compositions are known for use in delivering consumer-perceivable (e.g. sensory or visual) benefits to the skin. Many of such compositions attempt to achieve consumer-perceivable benefits by depositing benefit or active agents, including emollient oils, lipids, active beads, insoluble inorganic particles to the skin. Applicants have recognized, however, that the incorporation of such materials into an aqueous composition tends to result in undesirable phase separation of the benefit agents from the aqueous phase.

Various methods have been attempted to deal with phase separation problems, including the use of structured surfactant systems, inorganic silicates, and organic polymers, see, for example, US2009/0005449 A1, WO 2008/023145, and JP 4182348. However, there is still a need for suspension systems utilizing agents that are based on natural polymers to provide stable suspension capabilities to aqueous compositions.

### SUMMARY OF THE INVENTION

In one aspect, the present invention provides compositions comprising an aqueous carrier, one or more surfactants, and hydrogel particles comprising one or more polysaccharides, wherein said composition does not show phase separation over a period of ten days at room temperature.

In another aspect, the present invention provides compositions comprising an aqueous carrier, about 3 wt. % or more of anionic surfactant, hydrogel particles comprising one or more polysaccharides selected from the group consisting of carrageenans, low acyl gellen gum, low methyoxyl pectin, and a mixture of two or more thereof, and one or more cross-linking agents, wherein said composition comprises from greater than zero to less than 0.8 wt. % of said polysaccharides and from about 0.5 wt. % to about 3 wt. % of said cross-linking agent.

In yet another aspect, the composition of the present invention comprises methods of depositing suspended material to the skin, hair and other epithelial tissues to cleanse such region and/or treat such region for any of a variety of conditions including, but not limited to, acne, wrinkles, dermatitis, dryness, muscle pain, itch, and the like.

In yet another aspect, the composition of the present invention provides methods of treating and/or cleansing the human body, more particularly skin, hair and other epithelial tissues, to improve appearance/texture of such region and/or to provide additional benefits such as conditioning, moisturizing, fairness, and or combinations two or more thereof.

Applicants have discovered unexpectedly that the compositions of the present invention comprising: a) one or more surfactants; and b) hydrogel particles comprising one or more polysaccharides tend to exhibit improved stability as compared to other comparable compositions. For example, as shown in the Examples, applicants have tested the stability of compositions of the present invention and comparable compositions for phase separation in accord with the Stability and/or Accelerated Stability Tests described herein. While the comparable compositions tended to show phase separation within about 3 days even at room temperature, the present compositions tended to be stable for greater than three times that period and at higher temperatures.

According to certain embodiments, the compositions of the present invention preferably exhibit phase stability, as measured in accord with the Stability Test herein, and/or beads stability for a period of at least ten days. In certain more preferred embodiments, the compositions of the present invention exhibit phase stability and/or beads stability, preferably phase stability and beads stability, for a period of at least one month, more preferably, at least three months, more preferably about 6 months or more.

### BRIEF DESCRIPTION OF THE DRAWING

FIG. 1 is a graphical depiction of the relative breaking strength as a function of wt % of cross-linking agent of a certain composition of the claimed invention.

### DESCRIPTION OF THE INVENTION

As used herein, unless otherwise specified, all percentages of ingredients in compositions are weight percentage of active/solid ingredients based on the total weight of composition.

Any of a variety of hydrogel particles may be suspended or dispersed within the aqueous compositions of the present invention. As will be recognized by those of skill in the art, the term “hydrogel” (also called aquagel) refers generally to, a network of oligomers or polymer chains that are water-insoluble, sometimes found as a colloidal gel in which water is the dispersion medium. In certain preferred embodiments, hydrogel particles may be formed by making a hydrogel and mixing the hydrogel into a carrier system to form hydrogel particles suspended or dispersed within the carrier system.

According to the present invention the hydrogel particles comprise one or more polysaccharides. Examples of suitable polysaccharides include polysaccharide gums, such as carageenans, gellan gum, pectin, arabic, trajacanth, karaya, shatti, locust bean, guar, psyllium seed, quince seed, agar, algin, furcellaran, larch gum, and the like, polysaccharide starches, such as carboxymethylstarch, hydroxyethylstarch, hydroxypropylstarch, and the like, polysaccharides derived from celluloses, inulins, as well as combinations of any two or more of such polysaccharides, and the like. Examples of certain preferred polysaccharides include carageenans, such as k-carageenan and i-carrageenan, as well as, low acyl gellan gum, low methoxyl gelatin, and combinations of two or more of such polysaccharides and the like. According to certain more preferred embodiments, the polysaccharide comprises k-carageenan, i-carrageenan, or a combination thereof. Low acy gellan gum (sometimes called dacylated gellan gum) is well-known in the art and is typically manufactured through deacylation by treatment with alkali, see, e.g. Handbook of hydrocolloids, Edited by G. O. Phillips and P. A. Williams, 2000, Woodhead Publishing Limited, Section 7, pp. 118-135

The composition of the present invention may comprise any suitable amounts of polysaccharides. According to certain embodiments, the composition comprises from greater than zero to about 1 wt. % of polysaccharides. In certain preferred embodiments, the composition comprises from greater than zero to about 0.8 wt. % of polysaccharides, more preferably from about 0.1 to about 0.7 wt. %, more preferably from about 0.2 to about 0.6 wt. %, even more preferably from about 0.2 to about 0.5 wt. % of polysaccharides.

According to the present invention the hydrogel particles may also comprise one or more cross-linking agents. Examples of suitable cross-linking agents include metal salts or acids, such as salts of calcium, potassium, aluminum, sodium, magnesium, and the like. Examples of such salts include, for example, aluminium chloride, aluminium nitrate, aluminium sulphate, potassium chloride, calcium chloride or other calcium donor such as calcium gluconate, cement, sodium chloride, magnesium chloride, magnesium sulphate, potassium iodide, sodium hydrogen phosphate, magnesium nitrate, sodium nitrate, potassium nitrate, calcium nitrate, sodium silicate, mixtures of two or more thereof and the like. Certain preferred cross-linking agents include sodium chloride, calcium chloride, potassium chloride, or a mixture thereof. In certain particularly preferred embodiments, the cross-linking agent comprises potassium chloride.

The composition of the present invention may comprise any suitable amounts of cross-linking agent. According to certain embodiments, the compositions of the present invention comprise from greater than zero to about 3 wt. % of cross-linking agent, more preferably from about 0.05 to about 3 wt. %, more preferably about 0.1 to about 3 wt. %, and even more preferably, from about 0.2 to about 2 wt. % of cross-linking agent. In certain preferred embodiments, the composition comprising from about 0.5 to about 2.5 wt % of cross-linking agent.

The hydrogel particles dispersed within the composition of the present invention may be of any suitable size. According to certain preferred embodiments, the hydrogel particles have an average particle size of from about 1 micrometer (μm) to about 500 μm. In certain more preferred embodiments, the hydrogel particles in the composition have an average particle size of from about 1 μm to about 200 μm.

Any suitable surfactant may be used in the compositions of the present invention. Examples of suitable surfactants include anionic, non-ionic, and amphoteric surfactants.

As used herein, the term “anionic surfactant” refers to an ionic surfactant in which the hydrophilic portion of the surfactant carries negative charge. A description of anionic surfactants can be found in numerous texts and monographs, such as Rieger, Surfactant Encyclopedia, 2^{nd} Ed., C&T Ingredient Resource Series of COSMETICS AND TOILETRIES® magazine, published by Allured Publishing Corporation, Carol Stream, Ill. (1996), the relevant disclosures of which are incorporated by reference. Examples of suitable anionic surfactants include acylamino acids (and salts), such as acylgutamates, acyl peptides, sarcosinates, taurates, and the like; carboxylic acids (and salts), such as alkanoic acids (and alkanoates), ester carboxylic acids, ether carboxylic acids, and the like; phosphoric acid esters (and salts); sulfonic acids (and salts), such as acyl isethionates, alkyl isethionates, alkylaryl sulfonates, alkyl sulfonates, alkyl sulfosuccinates (and salts), and the like; and sulfuric acid esters, such as alkyl ether sulfates, alkyl sulfates, and the like. Examples of certain preferred anionic surfactants include alkyl sulfates; alkyl ether sulfates; alkyl monoglyceryl ether sulfates; alkyl monoglyceride sulfates; alkyl monoglyceride sulfonates; alkyl sulfonates; alkylaryl sulfonates; alkyl sulfosuccinates; alkyl ether sulfosuccinates; alkyl sulfosuccinamates; alkyl amidosulfosuccinates; alkyl carboxylates; alkyl amidoethercarboxylates; alkyl succinates; fatty acyl sarcosinates; fatty acyl amino acids; fatty acyl taurates; fatty alkyl sulfoacetates; alkyl phosphates, alkyl and acyl isethionates; and mixtures of two or more thereof. In certain more preferred embodiments, the anionic surfactants comprise sodium laureth sulfate, sodium lauryl sulfate, ammonium lauryl sulfate, ammonium laureth sulfate, sodium alpha-olefin sulfonate, sodium cocoyl isethionate, disodium laureth sulfosuccinate, sodium laurenth-13 carboxylate.

Any suitable amounts of anionic surfactant may be used in accord with the present invention. In certain embodiments, the compositions comprise from greater than zero to about 30 wt. % of the composition of anionic surfactant. In more preferred embodiments, the compositions comprise from about 1 to about 30 wt. %, more preferably about 3 to about 20 wt. %, and even more preferably from about 3 to about 15 wt. % of anionic surfactant (active solids).

As used herein, the term “amphoteric” means: 1) molecules that contain both acidic and basic sites such as, for example, an amino acid containing both amino (basic) and acid (e.g., carboxylic acid, acidic) functional groups; or 2) zwitterionic molecules which possess both positive and negative charges within the same molecule. The charges of the latter may be either dependent on or independent of the pH of the composition. Examples of zwitterionic materials include, but are not limited to, alkyl betaines and amidoalkyl betaines. The amphoteric surfactants are disclosed herein without a counter ion. One skilled in the art would readily recognize that under the pH conditions of the compositions of the present invention, the amphoteric surfactants are either electrically neutral by virtue of having balancing positive and negative charges, or they have counter ions such as alkali metal, alkaline earth, or ammonium counter ions. Examples of amphoteric surfactants suitable for use in the present invention include, but are not limited to amphocarboxylates such as alkylamphoacetates (mono or di); alkyl betaines; amidoalkyl betaines; amidoalkyl sultaines; amphophosphates; phosphorylated imidazolines such as phosphobetaines and pyrophosphobetaines; carboxyalkyl alkyl polyamines; alkylimino-dipropionates; alkylamphoglycinates (mono or di); alkylamphoproprionates (mono or di),); N-alkyl β-aminoproprionic acids; alkylpolyamino carboxylates; and mixtures of two or more thereof. Examples of certain preferred amphoteric surfactants include alkyl betaines, amidoalkylbetaines, phosophobetaines, alkylamphoglycinates, and combinations of two or more thereof. In certain more preferred embodiments, the amphoteric surfactant is selected from the group consisting of cocamidopropylbetaine, lauramidopropyl betaine, decyl betaine, lauryl betaine and combinations of two or more thereof.

Any suitable amounts of amphoteric surfactant may be used in accord with the present invention. In certain embodiments, the compositions comprise from zero to about 20 wt. % of the composition of amphoteric surfactant. In more preferred embodiments, the compositions comprise from about 1 to about 20 wt. %, more preferably about 1 to about 10 wt. %, and even more preferably from about 1 to about 5 wt. % of amphoteric surfactant (active solids).

As used herein, the term “nonionic surfactant” refers to an ionic surfactant in which the hydrophilic portion of the surfactant carries no charge. One class of nonionic surfactants useful in the present invention are polyoxyethylene derivatives of polyol esters, wherein the polyoxyethylene derivative of polyol ester (1) is derived from (a) a fatty acid containing from about 8 to about 22, and preferably from about 10 to about 14 carbon atoms, and (b) a polyol selected from sorbitol, sorbitan, glucose, α-methyl glucoside, polyglucose having an average of about 1 to about 3 glucose residues per molecule, glycerine, pentaerythritol and mixtures thereof, (2) contains an average of from about 10 to about 120, and preferably about 20 to about 80 oxyethylene units; and (3) has an average of about 1 to about 3 fatty acid residues per mole of polyoxyethylene derivative of polyol ester. Another class of suitable nonionic surfactants includes long chain alkyl glucosides or polyglucosides, which are the condensation products of (a) a long chain alcohol containing from about 6 to about 22, and preferably from about 8 to about 14 carbon atoms, with (b) glucose or a glucose-containing polymer. The alkyl glucosides have about 1 to about 6 glucose residues per molecule of alkyl glucoside. Examples of certain preferred nonionic surfactants include PEG-80 sorbitan laurate and Polysorbate 20. PEG-80 sorbitan laurate, which is a sorbitan monoester of lauric acid ethoxylated with an average of about 80 moles of ethylene oxide, is available commercially from ICI Surfactants of Wilmington, Del. under the trade name, “Atlas G-4280.” Polysorbate 20, which is the laurate monoester of a mixture of sorbitol and sorbitol anhydrides condensed with approximately 20 moles of ethylene oxide, is available commercially from ICI Surfactants of Wilmington, Del. under the trade name “Tween 20”.

Any suitable amounts of nonionic surfactant may be used in accord with the present invention. In certain embodiments, the compositions comprise from zero to about 20 wt. % of the composition of nonionic surfactant. In more preferred embodiments, the compositions comprise from about 1 to about 20 wt. %, more preferably about 1 to about 10 wt. %, and even more preferably from about 1 to about 5 wt. % of nonionic surfactant (active solids).

Other surfactants which can be utilized in the present invention are set forth in WO 99/21530, U.S. Pat. No. 3,929,678, U.S. Pat. No. 4,565,647, U.S. Pat. No. 5,720,964, and U.S. Pat. No. 5,858,948. According to certain preferred embodiments, the compositions of the present invention comprise at least one anionic surfactant. In certain other preferred embodiments, the compositions of the present invention comprise at least one anionic surfactant and at least one amphoteric surfactant.

Applicants have recognized that the compositions of the present invention tend to be stable compositions, preferably microgel compositions, that exhibit good ability to suspend particles and other materials therein. Accordingly, in certain preferred embodiments, the compositions of the present invention further comprise particles suspended therein. Examples of particles suitable for use in the present compositions include inert or active agents in the form of oils, lipids, or other fluid particles, as well as, beads or other solid particles, including those comprising synthetic polymers such as polyethylene, polystyrene, poly gelatins, arabic gums, collagens, polypeptides from vegetable or animal origin, alginates, polyamides, glycosamino glycans, mucopolysaccharides, ethylcellulose, titanium dioxide, mica, wax beads, silica, aluminum oxide, zinc oxide, titanium oxide, polyethylene oxide, talc, hydrocarbon, olive oil castor oil, sunflower oil, vaseline, coconut oil silicone oil, actives such as UV absorbers, pH modifiers, preservatives, odor absorbers, viscosity modifiers, neutralizers, antibacterial agents, botanical extracts, skin conditioners, moisturizers, skin fairness agents, an anti-acne agents, antioxidants, and or combinations two or more thereof, and the like. Examples of certain preferred particulate materials include titanium dioxide, mica, wax beads, silica, aluminum oxide, zinc oxide, titanium oxide, polyethylene oxide, talc, and combinations of two or more thereof.

Generally, particulate materials are supplied commercially with a wide distribution of sizes. In certain embodiments, particulate materials suitable for use herein comprise diameters of from about 0.01 to about 2000 micron. In certain preferred embodiments, the particulate materials have diameters of from about 0.1 to about 1000 micron, and even more preferably from about 1 to about 500 micron.

Any suitable amount of particulate materials may be used in the composition of the present invention. Preferably, the present compositions comprise from about 0.01 wt. % to about 10 wt. %, more preferably 0.05 wt. % to 8 wt. %, and most preferably from 0.05 wt. % to 5 wt. % of particulate materials.

Optional other ingredients may be incorporated into the composition of this invention (as particles or otherwise). Examples of such ingredients include pearlescent or opacifying agents, thickening agents, secondary conditioners, humectants, chelating agents, and additives which enhance the appearance, feel and fragrance of the compositions, such as colorants, fragrances, preservatives, pH adjusting agents, and the like.

The compositions of the present invention may be of any suitable yield stress for use in the present invention. According to certain preferred embodiments, the present compositions have a yield stress value, as measured in accord with the Yield Stress Test described below, of about 1 Pa or greater. In more preferred embodiments, the compositions have a yield value of about 1 to about 100 Pa, more preferably about 2 to about 100 Pa, even more preferably about 5 to about 100 Pa, and even more preferably about 10 to about 100 Pa.

The compositions of the present invention may be of any suitable viscosity. According to certain preferred embodiments, the present compositions have a viscosity of about 100-500,000 cps.

The compositions of the present invention may be made via a variety of conventional techniques. For example, in certain embodiments, the composition is made by adding the various ingredients and mixing. In certain embodiments, the various ingredients may be mixed in separate batches and the separate batches then combined. The combination of materials in batches or together may be done in the presence of heating where necessary. According to certain preferred embodiments, the compositions of the present invention are made by (a) combining water and surfactants into a premix, (b) combining water, polysaccharides and cross-linking agents (when necessary) with heating to from a main batch; (c) cooling the main batch and then combining the premix and the main batch and mixing to uniformity; and (d) optionally adding particles, if any, to the composition and mixing to uniformity.

The composition of the present invention may be used on the human body for a variety of end benefits. For example, the compositions may be applied topically to the skin, hair and other epithelial tissues to cleanse such region and/or treat such region for any of a variety of conditions including, but not limited to, acne, wrinkles, dermatitis, dryness, muscle pain, itch, and the like.

The composition of the present invention provides methods of treating and/or cleansing the human body more particularly skin, hair and other epithelial tissues to improve appearance/texture of such region and provide additional benefits such as conditioning, moisturizing, fairness, sunblock, and/or combinations two or more benefits thereof.

### EXAMPLES

The following examples will more fully illustrate the embodiments of this invention. The examples are provided for illustrative purposes and should not be considered as limiting the scope of the invention.

The sources for the materials utilized (and the active weight % of solids therein if less than 100%) in the following examples were as follows:

i-carrageenan was obtained from CPKelco under the tradename Genugel Carrageenan Cl123; k-carrageenan was obtained from CPKelco under the tradename Genugel Carrageenan Cl102; Mixture of methylchloroisothiazoline (1.125%) and methylisothiazoline (0.375%) was obtained from Rohm and Haas Company, Inc. under the tradename Kathon CG; Cocamidopropyl Betaine was obtained from Evonik Industries under the tradename TEGO® Betain F 50J (37% solids); Sodium laureth sulfate was obtained from Cognis under the tradename TEXAPON N70 LS-J (70% solids); deacylated gellen gum was obtained from CPkelco under the tradename KelcoGel CG-LA; silicon dioxide was obtained from Presperse LLC under the tradename Spheron L1500; dimethicone was obtained from Dow Corning under the tradename DC200. Water used is dionized (DI) water.

### Yield Stress Test:

To determine the Yield Stress for the composition of the present invention following test is performed:

Samples were placed in a water bath set at 25° C. for a period time sufficient to allow the sample to equilibrate (at least about an hour). 1.5 grams of the composition is taken from the samples and placed on the base plate of an advanced Rheometer AR 2000 having a 40 mm cone with a 2 degree angle, a 20 mm plate, a water bath, and a solvent trap. The sample size was just sufficient to allow some minor flow of the sample out of the gap once the final position of the cone and plate was reached (0.005 mm). To minimize shearing of the sample prior to testing, each sample was applied to the plate in a consistent manner, by gently scooping out the sample in one motion without significant shear or spreading, evenly layered on the plate, and without compressing and rotating the spatula away from the sample. The sample was centered on the base plate and laid relatively even across the plate. Once the measurement position was reached, a small bulge of the sample material protruded from the gap. This was removed quickly and gently so as not to disturb the top plate and pre-shear the sample. (If the top plate was moved then the run was aborted.) The sample is maintained at 25° C. for 5 minutes to have pre-equilibrium. The instrument was set for a controlled shear rate run (log) with a shear rate spanning from 0.01⁻¹, to 1000⁻¹, the output device attached to the Rheometer was set to plot stress (Pa) as a function of shear rate S⁻¹. Yield stress was determined from the plot of yield stress versus shear rate as the stress at which the curve departs from linearity. The average and standard deviation of the 2 runs were determined.

### Particle Size Test Method:

Particle size was measured by laser-diffraction-and-scattering type device MS2000 obtained from Malvern Instruments (China) using the following test methods:

Testing of a blank or reference standard: 80 ml of purified water taken in a 150 ml glass container and tested using MS 2000 instrument.

Preparation of a sample solution and testing: A sample solution is prepared by mixing 20 ml of the composition (gel solution) of the present invention with 60 ml purified water, the resulting solution is then stirred at 1800 rpm until uniform, then 80 ml of sample is taken in 150 ml beaker and tested using MS 2000 instrument.

### Phase Stability Test:

To determine Phase Stability the following test was performed: 150 g of a sample is taken in a 150 ml transparent plastic container/bottle and placed in a stability room kept at 25° C., which is considered as room temperature. The physical condition of the sample was inspected every 24 hours for up to at least 1 month or until observed phase separation. If phase separation was observed, the time of such observation was recorded.

### Accelerated Phase Stability Test:

For Accelerated Phase Stability, the following test was performed: for test at 50° C., 150 g of a sample is taken in a 150 ml transparent plastic container/bottle and placed in a stability oven (commercially available from Binder, model no. BF115) kept at 50° C.; for test at 40° C., 150 g of a sample is taken in a 150 ml transparent plastic container/bottle and placed in a stability oven kept at 40° C. The physical condition of the sample was inspected every 24 hours for up to at least 1 month or until phase separation is observed. If phase separation was observed, the time of such observation was recorded.

### FORMULATION EXAMPLES

### Example 1

The composition of example 1 was prepared according to the materials and amounts listed in Table 1 in accord with following steps:

Step I—Preparation of a Premix:

In a manufacturing vessel of 1 kg size, Deionized water and Texapon N70 were mixed together until uniform. Tego Betain F50J was added with a constant stirring to obtain a uniform solution/mixture/preparation.

Step II—Main Batch Preparation:

In a manufacturing vessel of 1 kg size, Deionized water and Genugel Carrageenan CI 102 were mixed together at a temperature in the range of from about 70° C. to 90° C. until the Genugel Carrageenan CI 102 dissolved, Potassium chloride was added to the reaction mixture with constant stirring to obtain a uniform mixture (solution/mixture/preparation). The resulting mixture was then cooled to room temperature under constant stirring or homogenizing using IKA-Werke ultra-turrax T25 Basic at speed 4 setting for 10 min to get a uniform mixture.

Step III—Combining the Premix and the Main Batch:

The premix was added to the resulting mixture obtained at the end of step II with a constant stirring to obtain a uniform solution/mixture/preparation.

Step IV—Adding Functional Beads:

To the resulting mixture of step III, Accuscrub GN207, P-SiO2-T (Silicon dioxide) and WS-Cwumm were added with a constant stirring until the mixture was uniform.

TABLE 1

| | | Concentration |
|---|---|---|
| Trade name | INCI name | (Wt/%) |
| Deionized Water | Water | 77.8 |
| Genugel Carrageenan | k-Carrageenan | 0.4 |
| CI 102 | | |
| Potassium chloride | Potassium chloride | 1 |
| Texapon N70 | Sodium laureth sulfate | 13 |
| Tego Betain F50J | Coco Betaine | 8 |
| Accuscrub GN207 | Polyethylene | 0.1 |
| P SiO2-T | Silicon dioxide | 0.1 |
| WS-Cwumm | Copernicia cerifera wax | 0.1 |
| | and ultramarines | |

### Examples 2-7

The composition of examples 2 through 7 were prepared according to the materials and amount listed in Table 2 in accord with following steps:

Step I—Preparation of a Premix:

In a manufacturing vessel of 1 kg size, Deionized water and Texapon N70 were mixed together until uniform. Tego Betain F50J was added with a constant stirring to obtain a uniform solution/mixture/preparation.

Step II—Main Batch Preparation:

In a manufacturing vessel of 1 kg size, Deionized water and Genugel Carrageenan CI 102 were mixed together at a temperature in the range of from about 70° C. to 90° C. until the Genugel Carrageenan CI 102 dissolved. Potassium chloride was added to the reaction mixture with a constant stirring to obtain a uniform mixture (solution/mixture/preparation). The resulting mixture was then cooled to room temperature under constant stirring or homogenizing using IKA-Werke ultra-turrax T25 Basic at speed 4 setting for 10 min to get a uniform mixture.

Step III—Combining the Premix and the Main Batch:

The premix was added to the resulting mixture obtained at the end of step II with a constant stirring to obtain a uniform solution/mixture/preparation.

Step IV—Adding Preservative:

To the resulting mixture of step III, Kathon CG was added with a constant stirring until the mixture was uniform.

The compositions of Table 2 were tested for yield stress by using the Yield Stress test method as mentioned in this description.

TABLE 2

| | | Concentration (Wt/%) | | | | | |
|---|---|---|---|---|---|---|---|
| Trade name | INCI name | Ex. 2 | Ex. 3 | Ex. 4 | Ex. 5 | Ex. 6 | Ex. 7 |
| Deionized Water | Water | 82.12 | 80.42 | 78.42 | 81.37 | 81.12 | 78.12 |
| Genugel | k-Carregeenan | 0.5 | 0.5 | 0.5 | 0.05 | 0.3 | 0.8 |
| Carrageenan CI | | | | | | | |
| 102 | | | | | | | |
| Potassium | Potassium chloride | 0.3 | 2 | 4 | 1.5 | 1.5 | 4 |
| chloride | | | | | | | |
| Kathon CG | Methylchloroisothiazoline (1.125%) | 0.08 | 0.08 | 0.08 | 0.08 | 0.08 | 0.08 |
| | and Methylisothiazoline (0.375%) | | | | | | |
| Tego Betain | Cocamidopropyl Betaine | 5 | 5 | 5 | 5 | 5 | 5 |
| F50J | | | | | | | |
| Texapon N70 | Sodium laureth sulfate | 12 | 12 | 12 | 12 | 12 | 12 |

| | | | | | | |
|---|---|---|---|---|---|---|
| Yield stress (Pa) | 1.6 | 25.1 | 20 | 0.8 | 6.3 | 25 |

### Example 8-13

The composition of examples 8 through 13 were prepared according to the materials and amount listed in Table 3 in accord with following four steps:

Step I—Preparation of a Premix:

In a manufacturing vessel of 1 kg size, Deionized water and Texapon N70 were mixed together until uniform. Tego Betain F50J was added with a constant stirring to obtain a uniform solution/mixture/preparation.

Step II—Main Batch Preparation:

In a manufacturing vessel of 1 kg size, Deionized water and Genugel Carrageenan CI 123 were mixed together at a temperature in the range of from about 70° C. to 90° C. until the Genugel Carrageenan CI 102 dissolved. Potassium chloride was added to the reaction mixture with a constant stirring to obtain a uniform mixture (solution/mixture/preparation). The resulting mixture was then cooled to room temperature under constant stirring or homogenizing using IKA-Werke ultra-turrax T25 Basic at speed 4 setting for 10 min to get an uniform mixture.

Step III—Combining the Premix and the Main Batch:

The premix was added to the resulting mixture obtained at the end of step II with a constant stirring to obtain a uniform solution/mixture/preparation.

Step IV—Adding Preservative:

To the resulting mixture of step III, Kathon CG was added with a constant stirring until the mixture was uniform.

The compositions of Table 3 were tested for yield stress by using the Yield Stress test method as mentioned in this description.

TABLE 3

| | | Concentration (Wt/%) | | | | | |
|---|---|---|---|---|---|---|---|
| Trade name | INCI name | Ex. 8 | Ex. 9 | Ex. 10 | Ex. 11 | Ex. 12 | Ex. 13 |
| Deionized | Water | 82.12 | 80.42 | 78.42 | 81.37 | 81.12 | 78.12 |
| Water | | | | | | | |
| Genugel | i-Carregeenan | 0.5 | 0.5 | 0.5 | 0.05 | 0.3 | 0.8 |
| Carrageenan | | | | | | | |
| CI 123 | | | | | | | |
| Potassium | Potassium chloride | 0.3 | 2 | 4 | 1.5 | 1.5 | 4 |
| chloride | | | | | | | |
| Kathon CG | Methylchloroisothiazoline (1.125%) | 0.08 | 0.08 | 0.08 | 0.08 | 0.08 | 0.08 |
| | and Methylisothiazoline (0.375%) | | | | | | |
| Tego Betain | Cocamidopropyl Betaine | 5 | 5 | 5 | 5 | 5 | 5 |
| F50J | | | | | | | |
| Texapon N70 | Sodium laureth sulfate | 12 | 12 | 12 | 12 | 12 | 12 |

| | | | | | | |
|---|---|---|---|---|---|---|
| Yield stress (Pa) | 0.6 | 7.9 | 3.9 | 0.3 | 0.9 | 5 |

### Examples 14-15

The composition of examples 14 through 15 were prepared according to the materials and amount listed in Table 4 in accord with the following steps:

Step I—Preparation of a Premix:

In a manufacturing vessel of 1 kg size, Deionized water and Texapon N70 were mixed together until uniform. Tego Betain F50J was added with a constant stirring to obtain a uniform solution/mixture/preparation.

Step II—Main Batch Preparation:

In a manufacturing vessel of 1 kg size, Deionized water and polysaccharides were mixed together at a temperature in the range of from about 70° C. to 90° C. until the Genugel Carrageenan CI 102 dissolved. Potassium chloride was added to the reaction mixture with a constant stirring to obtain a uniform mixture (solution/mixture/preparation). The mixture was then cooled to room temperature and IKA-Werke ultra-turrax T25 Basic was added with constant stirring (speed 4) for 10 min to homogenize the mixture.

For example 14, polysaccharides were the mixture of Genugel Carrageenan CI 102 and Kelcogel CG-LA, whereas for example 15, polysaccharides were the mixture of Genugel Carrageenan CI 102 and Low Methyoxyl Pectin.

Step III—Combining the Premix and the Main Batch:

The premix was added to the resulting mixture obtained at the end of step II with a constant stirring to obtain a uniform solution/mixture/preparation.

Step IV—Adding Preservative:

To the resulting mixture of step III, Kathon CG was added with a constant stirring until the mixture was uniform.

TABLE 4

| | | Concentration | |
|---|---|---|---|
| | | (Wt/%) | |
| Trade name | INCI name | Ex. 14 | Ex. 15 |
| Deionized Water | Water | 81.05 | 81.05 |
| Genugel Carrageenan | k-Carregeenan | 0.3 | 0.3 |
| CI 102 | | | |
| Kelcogel CG-LA | Low acyl gellen gum | 0.1 | |
| Low Methyoxyl Pectin | Low Methyoxyl Pectin | | 0.1 |
| Potassium chloride | Potassium chloride | 1.5 | 1.5 |
| Kathon CG | Methylchloroiso- | 0.05 | 0.05 |
| | thiazoline (1.125%) | | |
| | and Methyliso- | | |
| | thiazoline (0.375%) | | |
| Tego Betain F50J | Cocamidopropyl Betaine | 5 | 5 |
| Texapon N70 | Sodium laureth sulfate | 12 | 12 |

### Examples 16-19

The compositions of examples 16 through 19 were prepared according to the materials and amount listed in Table 5 in accord with the following steps:

Step I—Preparation of a Premix:

In a manufacturing vessel of 1 kg size, Deionized water and Texapon N70 were mixed together until uniform. Tego Betain F50J and Plantacare 2000UP were added with a constant stirring to obtain a uniform solution/mixture/preparation.

Step II—Main Batch Preparation:

In a manufacturing vessel of 1 kg size, Deionized water and Genugel Carrageenan CI 102 were mixed together at a temperature in the range of from about 70° C. to 90° C. until the Genugel Carrageenan CI 102 dissolved. Potassium chloride was added to the reaction mixture with a constant stirring to obtain a uniform mixture (solution/mixture/preparation). The resulting mixture was then cooled to room temperature under constant stirring or homogenizing using IKA-Werke ultra-turrax T25 Basic at speed 4 setting for 10 min to get a uniform mixture.

Step III—Combining the Premix and the Main Batch:

The premix was added to the resulting mixture obtained at the end of step II with a constant stirring to obtain a uniform solution/mixture/preparation.

Step IV—Adding Functional Beads:

To the resulting mixture of step III, DC 200 was added with a constant stirring until the mixture was uniform.

Step V—Adding Preservative:

To the resulting mixture of step IV, Kathon CG was added with a constant stirring until the mixture was uniform.

TABLE 5

| | | Concentration (Wt/%) | | | |
|---|---|---|---|---|---|
| Trade name | INCI name | Ex. 16 | Ex. 17 | Ex. 18 | Ex. 19 |
| Deionized | Water | 72.72 | 87.22 | 80.42 | 79.42 |
| Water | | | | | |
| Genugel | k-Carregeenan | 0.5 | 0.5 | 0.5 | 0.5 |
| Carrageenan | | | | | |
| CI 102 | | | | | |
| Potassium | Potassium | 2 | 2 | 2 | 2 |
| chloride | chloride | | | | |
| DC 200 | Dimethicone | 2 | 2 | 2 | 2 |
| Kathon CG | Methylchloroiso- | 0.08 | 0.08 | 0.08 | 0.08 |
| | thiazoline (1.125%) | | | | |
| | and Methyliso- | | | | |
| | thiazoline (0.375%) | | | | |
| Plantacare | Decyl Glucoside | | | | 4 |
| 2000UP | | | | | |
| Tego Betain | Cocamidopropyl | 2.7 | 2.2 | | |
| F50J | Betaine | | | | |
| Texapon | Sodium laureth | 20 | 6 | 15 | 12 |
| N70 | sulfate | | | | |

### Comparative Examples C1-C6

Compositions C1-C6 were prepared with the materials and amounts listed in Table 6 in accord with the procedures described in JP 4182348 with the additional step of adding the indicated surfactants (Tego Betain F50J and/or Texapon N70) at the end with mixing.

TABLE 6

| | | Concentration (Wt/%) | | | | | |
|---|---|---|---|---|---|---|---|
| Trade name | INCI name | C1 | C2 | C3 | C4 | C5 | C6 |
| Deionized Water | Water | 69.47 | 83.97 | 73.44 | 88.14 | 81.14 | 80.34 |
| KelcoGel CG-LA | Deacylated gellen gum | 0.02 | 0.02 | 0.2 | 0.2 | 0.2 | 0.2 |
| Calcium chloride | Calcium chloride | | | 0.66 | 0.66 | 0.66 | 0.66 |
| Magnesium | Magnesium chloride | 0.01 | 0.01 | | | | |
| chloride | | | | | | | |
| Spheron L1500 | SiO2 | 0.5 | 0.5 | | | | |
| DC 200 | Dimethicone | | | 2 | 2 | 2 | 2 |
| Ethenol | Ethenol | 5 | 5 | 1 | 1 | 1 | 1 |
| Methyl paraben | Methyl paraben | 0.3 | 0.3 | | | | |
| 1,3-Butanediol | 1,3-Butanediol | 2 | 2 | | | | |
| Plantacare | Decyl Glucoside | | | | | | 4 |
| 2000UP | | | | | | | |
| Tego Betain F50J | Cocamidopropyl Betaine | 2.7 | 2.2 | 2.7 | 2.2 | | |
| Texapon N70 | Sodium laureth sulfate | 20 | 6 | 20 | 6 | 15 | 12 |

### Example 20

### Stability Testing

The stability of the compositions of Examples 1-19 and C1-C6 were tested in accord with the Stability or Accelerated Stability Test at the temperatures as indicated in Table 7. The results are also shown in Table 7. As shown in the Table, the compositions of the present invention tested showed stability at elevated temperatures for at least 1 month, whereas the comparable compositions showed separation at room temperature after three days.

TABLE 7

| Example No. | 1 | 2 | 3 | 4 | 5 |
|---|---|---|---|---|---|
| Test Results | Stable at | Stable at | Stable at | Stable at | Stable at |
| | 40 C. for | 50 C. for | 50 C. for | 50 C. for | 50 C. for |
| | 6 month | 1 month | 1 month | 1 month | 1 month |
| Example No. | 6 | 7 | 8 | 9 | 10 |
| Test Results | Stable at | Stable at | Stable at | Stable at | Stable at |
| | 50 C. for | 50 C. for | 50 C. for | 50 C. for | 50 C. for |
| | 1 month | 1 month | 1 month | 1 month | 1 month |
| Example No. | 11 | 12 | 13 | 14 | 15 |
| Test Results | Stable at | Stable at | Stable at | Stable at | Stable at |
| | 50 C. for | 50 C. for | 50 C. for | 50 C. for | 50 C. for |
| | 1 month | 1 month | 1 month | 1 month | 1 month |
| Example No. | 16 | 17 | 18 | 19 | C1 |
| Test Results | Stable at | Stable at | Stable at | Stable at | Show |
| | 40 C. for | 40 C. for | 40 C. for | 40 C. for | separation |
| | 1 month | 1 month | 1 month | 1 month | at RT after |
| | | | | | 3 days |
| Example No. | C2 | C3 | C4 | C5 | C6 |
| Test Results | Show | Show | Show | Show | Show |
| | separation | separation | separation | separation | separation |
| | at RT after | at RT after | at RT after | at RT after | at RT after |
| | 3 days | 3 days | 3 days | 3 days | 3 days |

### Example 21

### Particle Size

The particle size for certain composition of present invention samples from examples 3 through 7 and examples 14 through 15 were tested as per the Particle Size test, the results of which are summarized in the Table 8.

TABLE 8

| | Ex. 3 | Ex. 5 | Ex. 6 | Ex. 7 | Ex. 14 | Ex. 15 |
|---|---|---|---|---|---|---|
| Mean particle | 78 | 70 | 37 | 186 | 71 | 27 |
| size (um) | | | | | | |

### Example 22

### Breaking Strength

Applicants have measured the breaking strength as a function of cross-linking agent associated with a certain composition of the present invention as follows:

### Process of Making Gel to be Measured:

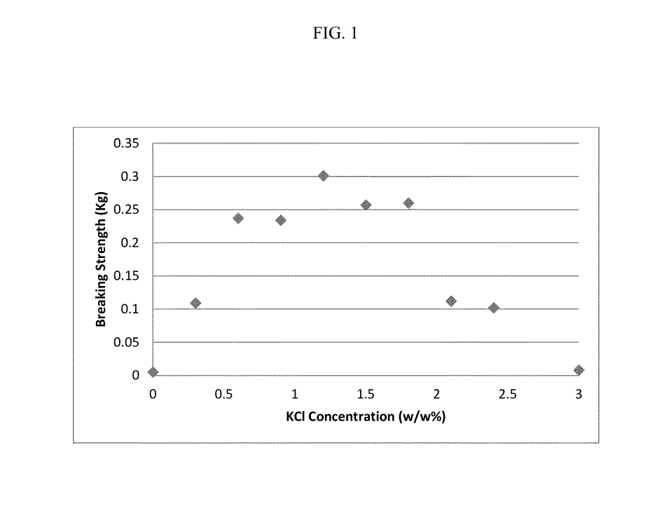

In a manufacturing vessel of 0.5 kg size, Deionized water and 0.5 wt % of Genugel Carrageenan CI 102 were mixed together at a temperature in the range of from about 70° C. to 90° C. until the Genugel Carrageenan CI 102 dissolved. Potassium chloride, in the amount indicated in FIG. 1 was added to the reaction mixture without stirring. The system was cooled to room temperature to form a gel.

The gel strength for each test sample was measured via a Standard test method used in food industry in which a TA.XTPlus Texture Analyzer with a 0.5 inch (1.27 cm) Radius Cylinder (P/0.5R) Cylinder probe is employed. The international standard test method named ISO 9665: 1998(E) can be used with the following settings: test mode is compression, pre-test speed is 0.5 mm/sec, test speed is 0.5 mm/sec, post-test speed is 0.5 mm/sec, target mode is distance, trigger type is force, trigger force is 5 g. Said ISO 9665: 1998(E) testing method, as described in International Method—Adhesives-Animal Glues-Methods of Sampling and Testing, ISO 9665, Second Edition (1998-09-15) is herein incorporated by reference. All gel strength measurements in this application were done with this method.

The results are shown graphically in FIG. 1. As illustrated, the gel breaking strength of the composition comprising Genugel Carrageenan CI 102 tested varies at different concentrations of potassium chloride. In the particular tested embodiment, preferred compositions having preferred stability and suspension properties are formed with potassium chloride concentrations of from about 0.3-2.5 wt %.

What is claimed is:

1. A composition comprising an aqueous carrier, hydrogel particles comprising one or more polysaccharides, and one or more surfactants, wherein said composition does not show phase separation over a period of ten days at room temperature as measured using the Phase Stability Test.

2. The composition of claim 1 wherein the polysaccharides are selected from the group consisting of carrageenan, low acyl gellen gum, low methyoxyl pectin or mixtures thereof.

3. The composition of claim 1 wherein the one or more polysaccharides comprise k-carrageenan.

4. The composition of claim 1 wherein the one or more polysaccharides comprise i-carrageenan.

5. The composition of claim 1, wherein the hydrogel particles comprise polysaccharide, cross-linking agent and water.

6. The composition of claim 5, wherein the cross-linking agent comprises a metal salt or metal acid.

7. The composition of claim 6, wherein the cross-linking agent comprises potassium chloride, calcium chloride, or a mixture thereof.

8. The composition of claim 7, wherein the cross-linking agent is potassium chloride.

9. The composition of claim 5 wherein the composition comprises from greater than zero to 1% of the one or more polysaccharides.

10. The composition of claim 5 wherein the composition comprises from 0.5 to 2.5% of the cross-linking agent.

11. The composition of claim 1 wherein the hydrogel particles have an average particle size of from 1 micrometer (μm) to 500 μm.

12. The composition of claim 1 wherein the one or more surfactants are selected from the group consisting of anionic, nonionic, amphoteric surfactants or combinations of two or more thereof.

13. The composition of claim 12 wherein the one or more surfactants comprise one or more anionic surfactants.

14. The composition of claim 13 wherein the one or more anionic surfactants comprise sodium laureth sulfate, sodium lauryl sulfate, ammonium lauryl sulfate, ammonium laureth sulfate, or a mixture of two or more thereof.

15. The composition of claim 14 wherein the one or more anionic surfactants comprise sodium laureth sulfate.

16. The composition of claim 12 wherein the one or more surfactants comprise one or more amphoteric surfactants.

17. The composition of claim 16 wherein the one or more amphoteric surfactants are selected from the group consisting of cocamidopropylbetaine, lauramidopropyl betaine, decyl betaine, lauryl betaine and combinations of two or more thereof.

18. The composition of claim 17 wherein the one or more amphoteric surfactants comprise cocamidopropylbetaine.

19. The composition of claim 1 further comprising one or more non-hydrogel particles dispersed therein.

20. The composition of claim 1 wherein the composition does not show phase separation over a period of one month at 50° C. as measured using the Accelerated Phase Stability Test.

21. A composition comprising an aqueous carrier, 3 wt. % on an active basis or more of anionic surfactant, hydrogel particles comprising one or more polysaccharides selected from the group consisting of carrageenans, low acyl gellen gum, low methyoxyl pectin or a mixture of two or more thereof, and from 0.5 to less than 3% of cross-linking agent, wherein said composition comprises from greater than zero to less than 0.8 wt. % on an active basis of polysaccharide selected from the group consisting of carrageenan, low acyl gellen gum, low methyoxyl pectin, and a mixture of two or more thereof.

22. A method of making a composition comprising hydrogel particles, the method comprising the steps of making a hydrogel comprising one or more polysaccharides, and mixing the hydrogel with an aqueous mixture comprising one or more surfactants to form a composition having an aqueous carrier, hydrogel particles comprising one or more polysaccharides dispersed therein, and one or more surfactants, wherein the resulting composition does not show phase separation over a period of ten days at room temperature as measured using the Phase Stability Test.

23. The method of claim 21 wherein the mixing step further includes mixing non-hydrogel particles into the aqueous mixture.
